(19) United States
(12) Patent Application Publication
(10) Pub. No.: US 2009/0112072 A1
(43) Pub. Date: Apr. 30, 2009

(54) SYSTEM THAT DISPLAYS BOTH VITAL SIGN INFORMATION AND ENTERTAINMENT CONTENT ON A COMMON VIDEO MONITOR

(75) Inventors: Matthew J. BANET, Del Mar, CA (US); Andrew S. TERRY, San Diego, CA (US); Marshall S. DHILLON, San Diego, CA (US); Thomas M. WATLINGTON, IV, La Mesa, CA (US)

Correspondence Address: WilmerHale/Triage Wireless, 60 State Street, Boston, MA, 02109, US

(73) Assignee: TRIAGE WIRELESS, INC., San Diego, CA (US)

(21) Appl. No.: 12/257,531

(22) Filed: Oct. 24, 2008

Related U.S. Application Data
(60) Provisional application No. 60/983,086, filed on Oct. 26, 2007.

(51) Int. Cl. A61B 5/021; A61B 5/02

(52) U.S. Cl. 600/301; 600/485

(57) ABSTRACT

A system for monitoring a patient's vital signs that includes: (1) a body-worn sensor unit containing a processor programmed to determine blood pressure information from the monitored vital signs and transmit that information via a wireless transceiver; (2) a monitor; and (3) a video display component. The monitor includes a display device, a wireless transceiver for receiving the blood pressure information, and a processor programmed to format that received information for display and to display a user interface for generating control information for the video display component. The video display component includes a display device, an interface for connecting to the external monitor interface, a computer network interface, a video input interface, and a processor programmed to respond to the control information from the external monitor by selecting whatever one or more of the monitor interface, the computer interface, and the video interface will provide information to be displayed.

This application claims the benefit of U.S. Provisional Application No. 60/983,086, filed Oct. 26, 2007, all of which is incorporated herein by reference.

### TECHNICAL FIELD

The present invention relates to medical devices for monitoring vital signs, e.g., blood pressure.

### BACKGROUND OF THE INVENTION

The prior art describes computer-based systems that monitor patients. These systems typically include a conventional vital sign monitor that can connect to an Internet-accessible computer. Typically the vital sign monitor includes: i) a cuff-based blood pressure measurement; ii) a system that measure an electrocardiograph (‘ECG’), heart rate, and respiratory rate; and iii) a pulse oximeter that measures blood oxygen saturation and an optical waveform called a plethysmograph (‘PPG’). In most cases the computer collects the vital signs measured by the monitor, avails them through the Internet to a web-based interface, and in some cases includes video conferencing hardware and software. With such a system, for example, a medical professional can remotely monitor an at-home patient. Patents that describe such systems include, for example: U.S. Pat. No. 5,434,611; U.S. Pat. No. 5,441,047; U.S. Pat. No. 5,902,234; and U.S. Pat. No. 5,919,141.

### SUMMARY OF THE INVENTION

The present system provides a patient-monitoring system which effectively monitors a patient and increases their comfort during, e.g., a hospital stay. The system features: i) a body-worn sensor featuring a continuous measurement of blood pressure and other vital signs; ii) a monitor, in wireless communication with the body-worn sensor, which receives the vital signs from the body-worn sensor; and iii) a video display monitor that interfaces with both the monitor and cable/Internet sources. During operation, the video display monitor renders vital signs measured by the body-worn sensor in addition to other content (e.g., television, Internet content, on-demand movies, games, and music videos). In this way the system continuously and cufflessly monitors the patient while simultaneously providing television and entertainment content. A single, large-area display renders vital signs, time-dependent ECG and PPG waveforms, along with video information.

Specifically, in one aspect, the system monitors a patient's vital signs with a sensor worn on the patient's body that continuously measures blood pressure information from a pulse transit time. The sensor features: i) an optical sensor attached to the patient and configured to generate time-dependent optical signal; ii) an electrode system attached to the patient and configured to generate a time-dependent electrical signal; and iii) a first processor configured to process the time-dependent optical and electrical signals with an algorithm to determine blood pressure information. The sensor additionally includes a first wireless transceiver that transmits the blood pressure information to a second wireless transceiver embedded within an external monitor. Through these transceivers the external monitor receives blood pressure information from the sensor. The monitor additionally includes a second processor that operates a user interface to generate control information for an external video display. The system also includes an external video display component featuring a monitor interface to the external monitor, a computer interface to a computer network, and a video interface to at least one other source for video content. The monitor interface receives blood pressure and control information from the monitor and, in response, displays the blood pressure information on the external video display component. The control information from the monitor commands the external video display to receive information from the computer network through the computer interface, and video information from the at least one other source for video content through the video interface.

In embodiments, the external video display component is a plasma, LCD, or projected display. The external monitor can also be configured to generate control information that commands the external video display component to display both blood pressure information and video information, e.g. images from a video conference. Typically the external monitor features a touchpanel display to render a graphical user interface, a video camera, and a barcode scanner. The barcode scanner reads barcodes worn by the patient (describing their demographic information), and adhered by the body-worn sensor (describing a media access control, or ‘MAC address’, of its internal Bluetooth transmitter). The monitor also includes wireless systems (e.g., Bluetooth, WiFi, and cellular modems) for sending information to external sources (e.g., a hospital IT system or central nursing station).

In embodiments, the video interface operating on the external video display includes an interface to a video conferencing service, a series of television stations, or a service that provides on-demand access to movies, games, and music.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic view of a multi-purpose system featuring a body-worn sensor and monitor that allows a hospitalized patient to be monitored and view content using a video display monitor.

FIG. 2 is a schematic view of the hospitalized patient of FIG. 1 wearing the body-worn sensor, which in turn communicates wirelessly with the monitor and video display monitor of FIG. 1.

FIG. 3 is a top, open view of the body-worn sensor of FIGS. 1 and 2.

FIG. 4 is a three-dimensional plan view of the monitor of FIGS. 1 and 2.

### DETAILED DESCRIPTION

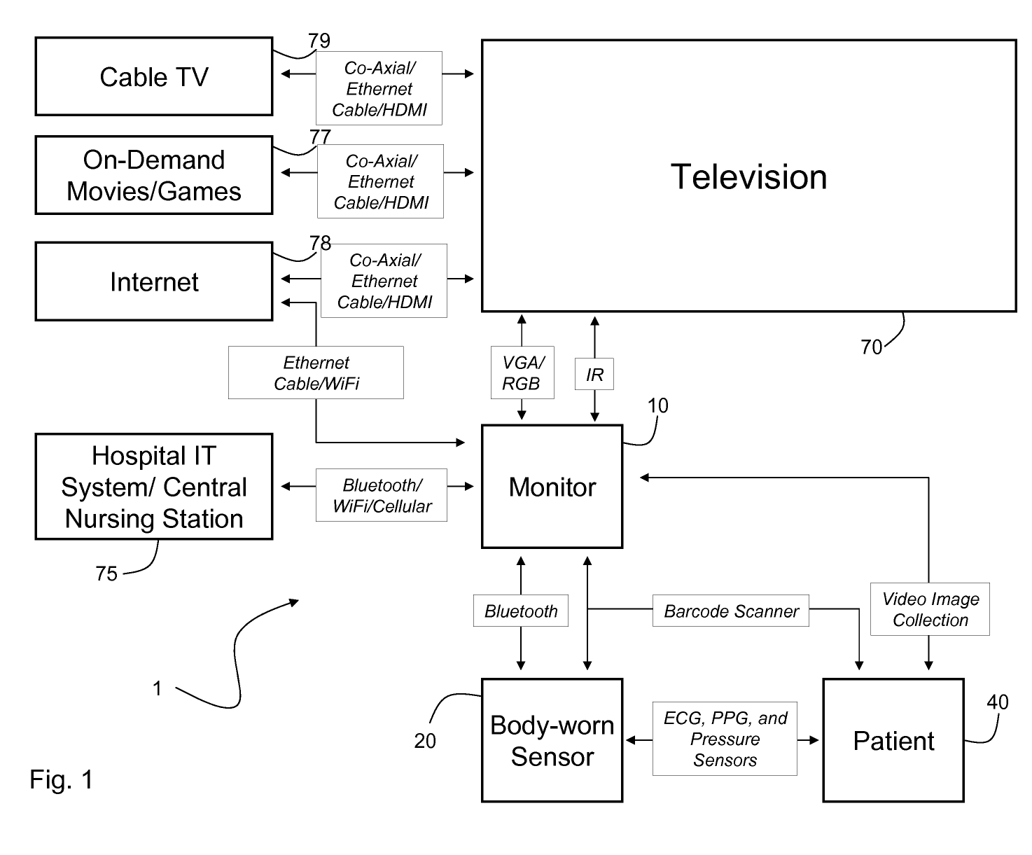

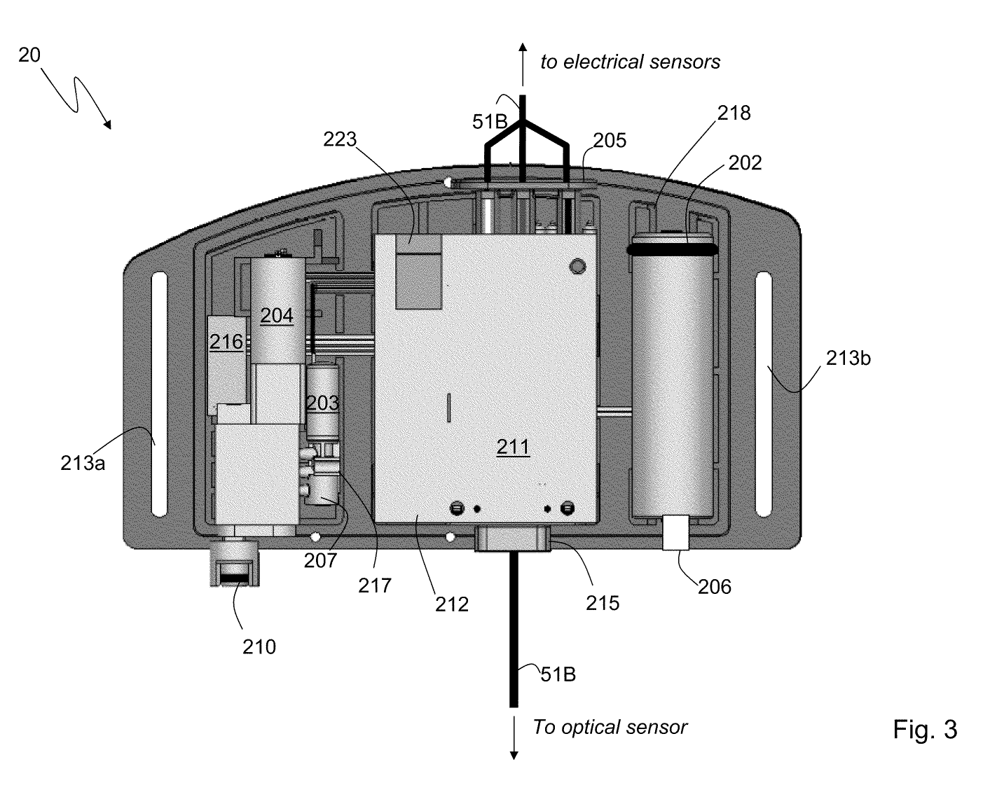

FIG. 1 shows a multi-purpose system 1 that monitors a patient's vital signs and additionally allows them to watch television, select movies on demand, play video games, access the Internet, and perform real-time video conferencing. The patient 40, for example, is located in a hospital room. The system 1 features a body-worn sensor 20 that attaches to the patient's right or left arm to measure vital signs (e.g., blood pressure, oxygen saturation, heart rate, respiratory rate, and temperature), waveforms (e.g. ECG and PPG), and other information (e.g. patient motion). Such a body-worn system is described, for example, in VITAL SIGN MONITOR MEASURING BLOOD PRESSURE USING OPTICAL, ELECTRICAL, AND PRESSURE WAVEFORMS (U.S. Ser. No. 12/138,194; filed Jun. 12, 2008). The body-worn sensor 20, which is described in more detail with reference to FIG. 3, features a series of optical, electrical, and pressure sensors that measure unique time-dependent waveforms from the patient 40. The body-worn sensor 20 includes a high-end microprocessor programmed to analyze the waveforms to determine the patient's vital signs, as described in more detail below.

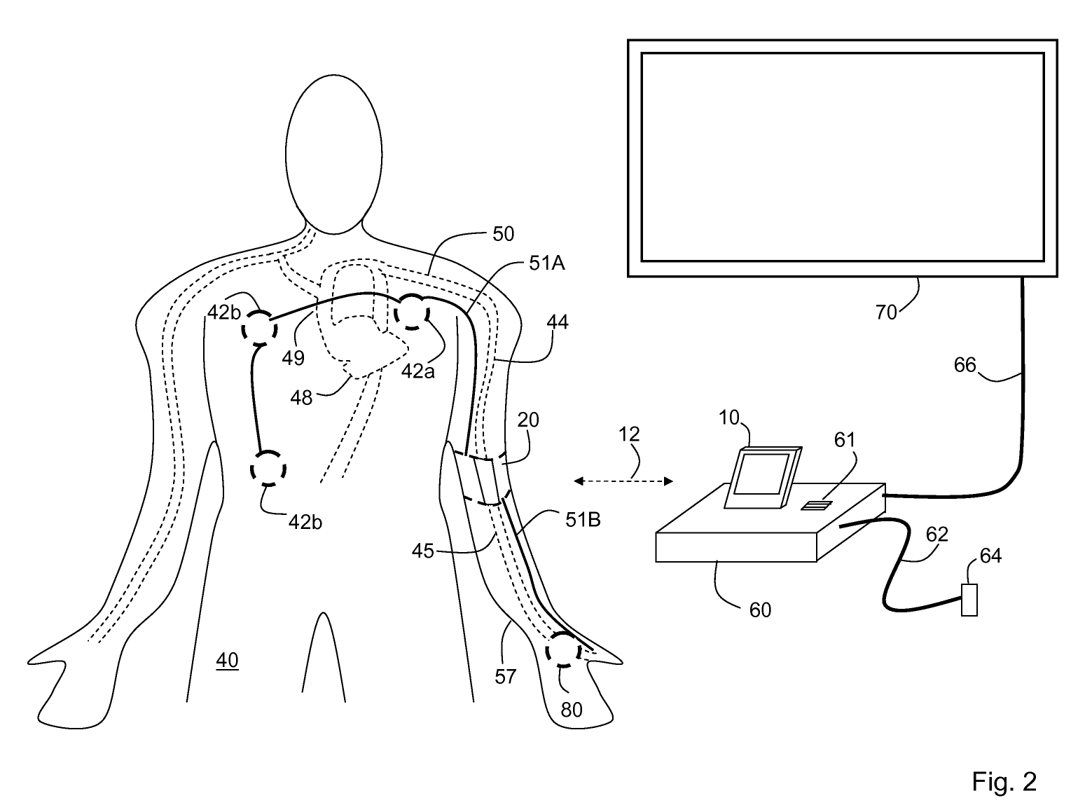

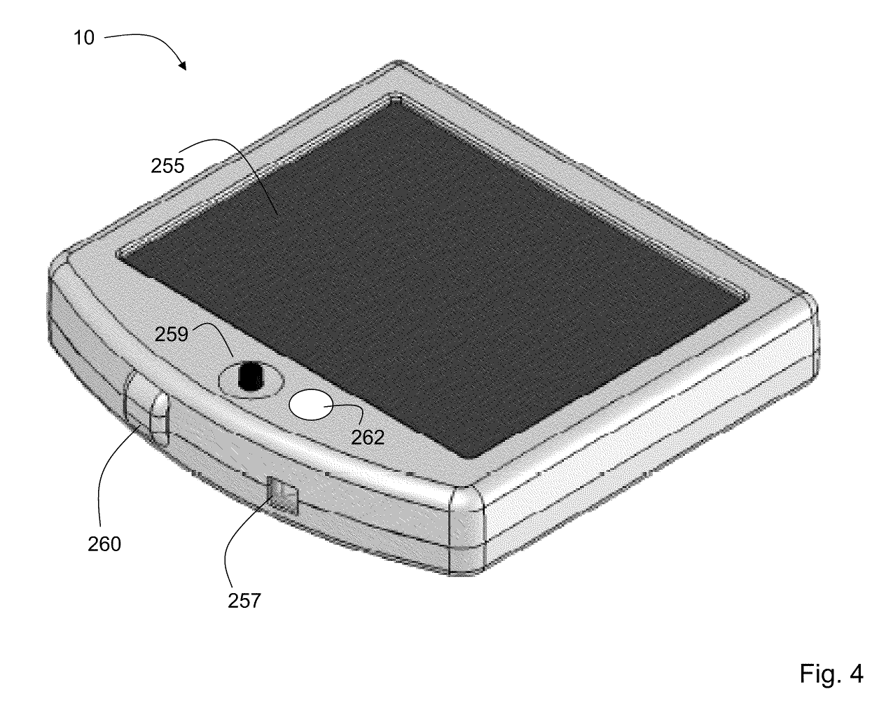

Once the body-worn sensor 20 measures the patient's vital signs, it transmits them through a wireless Bluetooth® interface to a monitor 10, which can be either hand-held or cradle-mounted. The monitor 10, which is described in more detail with respect to FIG. 4, includes a relatively small touchpanel display that renders the parameters it receives from the body-worn sensor 20, along with an icon-driven graphical user interface. So that vital signs and waveforms can be rendered on a larger, easily viewed display, the monitor 10 connects through a standard VGA/RGB interface to a wall-mounted television 70, e.g. an LCD or plasma television. These devices typically include standard video connectors on their back panels. Typically the hardware component of the VGA/RGB interface consists of a connector, mounted in a cradle similar to that shown in FIG. 2, which mates with a connector on monitor 10. The connector connects through a standard video cable to television 70. In this configuration, television 70 operates in a standard RGB mode to render vital signs and waveforms with a format dictated by the monitor 10. To control the television 70, e.g. to switch between display of vital signs and entertainment content, change channels, and adjust its volume, the monitor 10 can be programmed to render a simple, easy-to-read user interface on its touchpanel display that includes buttons and icons that allow a user to control the entertainment content rendered on the television 70. To operate in this mode, the monitor 10 additionally includes a conventional IR light-emitting diode (‘LED’) built into its top portion that is controlled by icons on the monitor's touchpanel and software running on a processor in the monitor. These systems modulate the blinking pattern (e.g. blinking frequency) of the IR LED to function as a conventional remote control. The blinking pattern is matched to the make and model of the particular television. Typically the monitor will include a variety of blinking patterns stored in a computer memory; the appropriate pattern can be selected through the monitor's touchpanel. In this configuration, for example, the monitor 10 can control the television 70 can also display: i) standard television programs which it receives through, e.g., a standard cable television system 79; ii) content which it receives from the Internet 78; iii) high-definition multimedia content; and, iv) on-demand movies and games, which it receives from a movie/game system 77. Standard co-axial, Ethernet cables, or High-Definition Multimedia Interface (HDMI) cables typically supply this content to the television 70.

The monitor 10 relays vital signs and other parameters (e.g. PPG and ECG waveforms) from the body-worn sensor 20 to the television 70. Using its internal Bluetooth transceiver, the monitor 10 can also send this information to a hospital IT system or central nursing station 75. For example, the monitor can transmit information over a Bluetooth ‘mesh’ network, or alternately through a conventional WiFi network (e.g. a network based on 802.11 protocol). This allows the hospital's medical professionals to monitor the patient 40 remotely. The wirelessly transmitted signal is typically sent to a matched transceiver that connects directly to the hospital IT system or central nursing station 75, or to an internal network including a series of wireless nodes that, in turn, connects to this system. In alternate embodiments, the monitor 10 includes secondary transmitters, e.g. cellular modems, which connect to the hospital IT system or central nursing station 75 through, respectively, local-area or wide-area networks.

The monitor 10 further includes a barcode scanner that allows it to scan a barcode on the body-worn sensor 20. The barcode includes, e.g., information on the body-worn sensor and the MAC address of its internal Bluetooth transmitter that, once processed by the monitor's internal microprocessor, allows the body-worn sensor 20 and monitor 10 to be effectively ‘paired’. This ensures that the monitor 10 and television 70 do not display information from a secondary body-worn sensor, e.g. one attached to a patient in a neighboring hospital room. The barcode scanner can also be used to scan a barcode worn on the patient's wrist which includes, e.g., personal and medical information, or medication prescribed to the patient.

The monitor 10 can further include a small video camera, mounted on its front surface, which collects video images of the patient 40. Using an Ethernet or wireless (e.g. WiFi) connection to the Internet 78, the monitor transmits images of the patient to video conferencing software located on a remote computer, where they are then viewed by an external person. Likewise, video images of the external person can be sent through the Internet 78 to the monitor 10, and from there through the VGA/RGB interface to the television 70, where they are viewed by the patient 40. This allows, e.g., the patient 40 to video conference with the external person. The external person can be, e.g., a medical professional in the hospital, or a family member at home.

FIG. 2 illustrates the above-mentioned system, featuring the monitor 10, body-worn sensor 20, and wall-mounted television 70. In a preferred embodiment, the body-worn sensor 20 makes a cuffless measurement of blood pressure, which is described in more detail in the following patent applications, the contents of which are incorporated by reference: This process is described in detail in the following co-pending patent applications, the contents of which are incorporated herein by reference: VITAL SIGN MONITOR MEASURING BLOOD PRESSURE USING OPTICAL, ELECTRICAL, AND PRESSURE WAVEFORMS (U.S. Ser. No. 12/138,194; filed Jun. 12, 2008); and, VITAL SIGN MONITOR FOR CUFFLESSLY MEASURING BLOOD PRESSURE CORRECTED FOR VASCULAR INDEX (U.S. Ser. No. 12/138,199; filed Jun. 12, 2008), describe these components in more detail. Specifically, to perform the cuffless blood pressure measurement, the body-worn sensor collects and analyzes time-dependent optical, electrical, and pressure waveforms from the patient 40, and analyzes them with a technique described in the above-mentioned patent applications to determine blood pressure and other vital signs.

The following summarizes this technique. During a measurement the patient's heart 48 generates electrical impulses that pass through the body near the speed of light. These impulses stimulate each heart beat, which in turn generates a pressure wave that propagates through the patient's vasculature at a significantly slower speed. Immediately after the heartbeat, the pressure wave leaves the aorta 49, passes through the subclavian artery 50, to the brachial artery 44, and from there through the radial artery 45 to smaller arteries in the patient's fingers. The body-worn sensor 20 attaches to the patient's arm 57. A three-patch electrode system 42a, 42b, 42c attached to the patients' chest and connects to the body-worn sensor 20 by a first cable 51A to measure unique electrical signals. These signals pass through the first cable 51A to an amplifier/filter circuit within the body-worn sensor 20. There, the signals are processed using the amplifier/filter circuit to determine an analog electrical signal, which is then digitized with a first channel on an analog-to-digital converter to form the electrical waveform, and finally stored in memory. The electrical waveform represents a single-lead ECG that features a sharp spike, called the ‘QRS complex’, for each heartbeat. Using a reflection-mode geometry, an optical sensor 80 attached to the body-worn sensor 20 measures an optical waveform from an arteries in the patient's wrist or hand. This signal passes through a second cable 51B to the body-worn sensor 20, where it is amplified using a second amplifier/filter circuit, and digitized with a second channel within the analog-to-digital converter. The digitized signal represents the optical waveform, which typically features a time-dependent ‘pulse’ corresponding to each heartbeat. Each pulse represents a volumetric change in an underlying artery caused by the propagating pressure wave.

The body-worn sensor 20 also includes a pneumatic pump-and-valve system, and attaches to the patient with an arm-worn band that includes an inflatable bladder. When the pump inflates the bladder, it imparts a time-dependent pressure to the patient's brachial artery 44 that affects the amplitude of the optical waveform and the time delay between the QRS complex in the electrical waveform, and the onset of the pulse in the optical waveform. At the same time, ‘pulsations’ in the patient's arm caused by the increased pressure couple into the bladder in the arm-worn band, and are measured by a pressure sensor in the body-worn sensor 20. This results in a series of pressure pulses that are mapped onto the pressure waveform. As described in the above-referenced patent applications, the microprocessor in the body-worn sensor 20 is programmed to process the time-dependent optical, electrical, and pressure waveforms to determine the patient's blood pressure and other vital signs. Measurements made in the presence of an applied pressure are described as ‘pressure-dependent measurements’, and determine systolic, diastolic, and mean arterial pressure. Once these parameters are determined, the body-worn sensor is programmed to use them and the same optical and electrical sensors to make continuous ‘pressure-free measurements’ using only the QRS complex in the ECG and the foot of the pulse in the PPG. There, the electrical signal is combined with those measured by other electrodes placed on the patient's body to determine an ECG which is digitized and processed with, respectively, the analog-to-digital converter and microprocessor. Using a technique referred to in the above-mentioned patent applications as the ‘composite measurement’, information derived from the electrical waveform is combined with information derived from the optical waveform to determine the patient's blood pressure and heart rate.

The above-described system can be used in a number of different settings, including both the home and hospital. A patient 40 in a hospital, for example, can continuously wear the body-worn sensor 20 over a time period ranging from minutes to several days. During this period, the body-worn sensor 20 is powered by a rechargeable battery, and continuously measures blood pressure and other vital signs using the technique described above. At a predetermined interval (typically, every few minutes) the sensor armband transmits this information through a short-range Bluetooth interface 12 to the monitor 10, which is typically seated in a cradle 60 next to a bed in the hospital. The cradle 60 includes a VGA/RGB connector (not shown in the figure) that mates with a connector on the bottom surface of the monitor 10 and sends signals through a cable 66 to the television 70. This allows the monitor 10 to be easily seen and controlled by the patient or caregiver, while also serving as a ‘hub’ that routes information measured by the body-worn sensor 20 to the television 70. The patient 40 or medical professional can tap icons on the monitor's graphical user interface to select modes where vital signs, television, Internet, or on-demand movies are displayed.

The cradle 60 additionally includes an AC adaptor 62 that plugs into a wall outlet 64 and continuously charges the monitor's battery as well as a spare battery 61 for the body-worn sensor 20. When the original rechargeable battery in the body-worn sensor 20 is depleted, the caregiver (or patient) 40 replaces it with the spare battery 61 in the cradle 60.

FIG. 3 shows a top view of the body-worn sensor 20 used to conduct the above-described measurements. The body-worn sensor 20 features a single circuit board 212 including connectors 205, 215 that connect through separate cables 51A, 51B to, respectively, electrodes worn on the patient's body and optical sensor worn on the patient's wrist. During both pressure-dependent and pressure-free measurements, these sensors measure electrical and optical signals that pass through the connectors 51A, 51B to discrete circuit components 211 on the bottom side of the circuit board 212. The discrete components 211 include: i) analog circuitry for amplifying and filtering the time-dependent optical and electrical waveforms; ii) an analog-to-digital converter for converting the time-dependent analog signals into digital waveforms; and a iii) microprocessor programmed to process the digital waveforms to determine blood pressure according to the above-described technique, along with other vital signs. The body-worn sensor 20 attaches to an arm-worn cuff using Velcro® through two D-ring loops 213a, 213b. The cuff secures the body-worn sensor 20 to the patient's arm.

To measure the pressure waveform during a pressure-dependent measurement, the circuit board 212 additionally includes a small mechanical pump 204 for inflating the bladder within the armband, and a solenoid value 203 for controlling the bladder's inflation and deflation rates. The pump 204 and solenoid valve 203 connect through a manifold 207 to a connector 210 that attaches through a tube (not shown in the figure) to the bladder in the armband, and additionally to a digital pressure sensor 216 that senses the pressure in the bladder. The solenoid valve 203 couples through the manifold 207 to a small ‘bleeder’ valve 217 featuring valve that controls air to slowly releases pressure or rapidly release pressure. Typically the solenoid valve 203 is closed as the pump 204 inflates the bladder. For measurements conducted during inflation, pulsations caused by the patient's heartbeats couple into the bladder as it inflates, and are mapped onto the pressure waveform. The digital pressure sensor 216 generates an analog pressure waveform, which is then digitized with the analog-to-digital converter described above. The microprocessor processes the digitized pressure, optical, and electrical waveforms to determine systolic, mean arterial and diastolic blood pressures. Once these measurements are complete, the microprocessor immediately opens the solenoid valve 203, causing the bladder to rapidly deflate.

Alternatively, for measurements done on deflation, the pump 204 inflates the bladder to a pre-programmed pressure above the patient's systolic pressure. Once this pressure is reached, the microprocessor opens the solenoid valve 203, which couples to the ‘bleeder’ valve 217 to slowly release the pressure. During this deflation period, pulsations caused by the patient's heartbeat are coupled into the bladder and are mapped onto the pressure waveform, which is then measured by the digital pressure sensor 215. Once the microprocessor determines systolic, mean arterial, and diastolic blood pressure, it opens the solenoid valve 203 to rapidly evacuate the pressure.

A rechargeable lithium-ion battery 202 mounts directly on the armband's flexible plastic backing 218 to power all the above-mentioned circuit components. Alternately, the armband's flexible plastic backing 218 additionally includes a plug 206 which accepts power from a wall-mounted AC adaptor. The AC adaptor is used, for example, when measurements are made over an extended period of time. A Bluetooth transmitter 223 is mounted directly on the circuit board 212 and, following a measurement, wirelessly transmits information to an external monitor. A rugged plastic housing (not shown in the figure) covers the circuit board 212 and all its components.

FIG. 4 shows a three-dimensional plan view of the monitor 10 that receives the Bluetooth-transmitted information from the body-worn sensor, and routes this information to the television. The front face of the monitor 10 includes a touchpanel display 255 that renders the icon-driven graphical user interface, a circular on/off button 259, and a CCD video camera 262. The CCD video camera 262 detects real-time digital images of the patient and sends them through the Internet as described above to an external computer system. A similar monitor has been described previously by Applicants in: BLOOD PRESSURE MONITOR (U.S. Ser. No. 11/530,076; filed Sep. 8, 2006) and MONITOR FOR MEASURING VITAL SIGNS AND RENDERING VIDEO IMAGES (U.S. Ser. No. 11/682,177; filed Mar. 5, 2007), the contents of which are incorporated herein by reference. The monitor 10 includes an internal Bluetooth transmitter (not shown in the figure) that can include an antenna 260 increase the strength of the received signal. To pair with a body-worn sensor, such as that shown in FIG. 3, the monitor 250 includes a barcode scanner 257 on its top surface. During operation, a user holds the monitor 10 in one hand, and points the barcode scanner 257 at a printed barcode adhered to the plastic cover surrounding the body-worn sensor. The user then taps an icon on the touchpanel display 255, causing the barcode scanner 257 to scan the barcode. The printed barcode includes information on the body-worn sensor's Bluetooth transceiver that allows it to pair with the monitor's Bluetooth transceiver. The scanning process decodes the barcode and translates its information to a microprocessor within the monitor 10. Once the information is received, software running on the microprocessor analyzes it to complete the pairing. This methodology forces the user to bring the monitor into close proximity to the body-worn sensor, thereby reducing the chance that vital sign information from another body-worn sensor is erroneously received and displayed.

In addition to those techniques described above, a number of additional techniques can be used to calculate blood pressure from the optical, electrical, and pressure waveforms. These are described in the following co-pending patent applications, the contents of which are incorporated herein by reference: 1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL SIGN MONITOR FOR ATHLETIC APPLICATIONS (U.S. Ser. No.; filed Sep. 13, 2004); 5) CUFFLESS BLOOD PRESSURE MONITOR AND ACCOMPANYING WIRELESS MOBILE DEVICE (U.S. Ser. No. 10/967,511; filed Oct. 18, 2004); 6) BLOOD PRESSURE MONITORING DEVICE FEATURING A CALIBRATION-BASED ANALYSIS (U.S. Ser. No. 10/967,610; filed Oct. 18, 2004); 7) PERSONAL COMPUTER-BASED VITAL SIGN MONITOR (U.S. Ser. No. 10/906,342; filed Feb. 15, 2005); 8) PATCH SENSOR FOR MEASURING BLOOD PRESSURE WITHOUT A CUFF (U.S. Ser. No. 10/906,315; filed Feb. 14, 2005); 9) PATCH SENSOR FOR MEASURING VITAL SIGNS (U.S. Ser. No. 11/160,957; filed Jul. 18, 2005); 10) WIRELESS, INTERNET-BASED SYSTEM FOR MEASURING VITAL SIGNS FROM A PLURALITY OF PATIENTS IN A HOSPITAL OR MEDICAL CLINIC (U.S. Ser. No. 11/162,719; filed Sep. 9, 2005); 11) HAND-HELD MONITOR FOR MEASURING VITAL SIGNS (U.S. Ser. No. 11/162,742; filed Sep. 21, 2005); 12) CHEST STRAP FOR MEASURING VITAL SIGNS (U.S. Ser. No. 11/306,243; filed Dec. 20, 2005); 13) SYSTEM FOR MEASURING VITAL SIGNS USING AN OPTICAL MODULE FEATURING A GREEN LIGHT SOURCE (U.S. Ser. No. 11/307,375; filed Feb. 3, 2006); 14) BILATERAL DEVICE, SYSTEM AND METHOD FOR MONITORING VITAL SIGNS (U.S. Ser. No. 11/420,281; filed May 25, 2006); 15) SYSTEM FOR MEASURING VITAL SIGNS USING BILATERAL PULSE TRANSIT TIME (U.S. Ser. No. 11/420,652; filed May 26, 2006); 16) BLOOD PRESSURE MONITOR (U.S. Ser. No. 11/530,076; filed Sep. 8, 2006); 17) TWO-PART PATCH SENSOR FOR MONITORING VITAL SIGNS (U.S. Ser. No. 11/558,538; filed Nov. 10, 2006); and, 18) MONITOR FOR MEASURING VITAL SIGNS AND RENDERING VIDEO IMAGES (U.S. Ser. No. 11/682,177; filed Mar. 5, 2007).

Other embodiments are also within the scope of the invention. For example, hardware components comparable to those described above can also be used with the monitor and body-worn sensor. For example, other wireless transceivers, e.g. Zigbee, part-15, or other low-power radios, can be used in place of Bluetooth. In addition, a variety of software configurations can be run on the monitor to give it a PDA-like functionality. These include, for example, Micro C OS®, Linux®, Microsoft Windows®, embOS, VxWorks, SymbianOS, QNX, OSE, BSD and its variants, FreeDOS, FreeRTOX, LynxOS, or eCOS and other embedded operating systems. The monitor can also run a software configuration that allows it to receive and send voice calls, text messages, or video streams received through the Internet or from the nation-wide wireless network it connects to. The bar-code scanner described with reference to FIG. 4 can also be used to capture patient or medical professional identification information, or other such labeling. It can be replaced with, e.g., a system for reading RFID tags. Information from these systems can be used, for example, to communicate with a patient in a hospital or at home. In other embodiments, the monitor can connect to an Internet-accessible website to download content, e.g., calibrations, software updates, text messages, and information describing medications, from an associated website. As described above, the monitor can connect to the website using both wired (e.g., USB port) or wireless (e.g., short or long-range wireless transceivers) means. It can include a software-driven keyboard and mouse. In still other embodiments, ‘alert’ values corresponding to vital signs and the pager or cell phone number of a caregiver can be programmed into the monitor using its graphical user interface. If a patient's vital signs meet an alert criteria, software on the device can send a wireless ‘page’ to the caregiver, thereby alerting them to the patient's condition. For additional patient safety, a confirmation scheme can be implemented that alerts other individuals or systems until acknowledgment of the alert is received.

The functionality described herein can be implemented by code executing on a processor. The code is typically stored on and read from a digital storage medium, such as RAM, ROM, a CD, etc.

Still other embodiments are within the scope of the following claims.

What is claimed is:

1. A system for monitoring a patient's vital signs, the system comprising:
a sensor unit to be worn on the patient's body to monitor vital signs of the patient, said sensor unit including a first wireless transceiver and a first processor programmed to determine blood pressure information from the monitored vital signs of the patient and transmit the blood pressure information via the first wireless transceiver;
an external monitor; and
an external video display component,
wherein the external monitor includes a first display device, a second wireless transceiver for receiving the blood pressure information from the sensor unit, and a second processor programmed to format the blood pressure information for display by the external video display component and further programmed to display on the first display device a user interface through which the patient generates control information for controlling the external video display component, and
wherein the external video display component includes a second display device, a monitor interface for connecting to the external monitor to receive the formatted blood pressure information, a computer interface for connecting to a computer network, a video interface for connecting to at least one other source for video content, and a third processor programmed to respond to the control information from the external monitor by selecting whatever one or more of the monitor interface, the computer interface and the video interface to provide information to be displayed on the second display device.

2. The system of claim 1, wherein the sensor unit comprises:
an optical sensor for attaching to the patient and generating a time-dependent optical signal representing a flow of blood within the patient; and
an electrode system for attaching to the patient and generating a time-dependent electrical signal representing activity of the patient's heart,
wherein the first processor is further programmed to process the time-dependent optical and electrical signals to determine blood pressure information.

3. The system of claim 1, wherein the external video display component comprises a plasma, LCD, or projected display.

4. The system of claim 1, wherein the second processor is further programmed to generate control information that commands the external video display component to display both blood pressure information and other video information from at least one of the computer interface and the video interface.

5. The system of claim 4, wherein the second processor is further programmed to generate control information that commands the external video display component to display both blood pressure information and images from a video conference.

6. The system of claim 1, wherein the first display device comprises a touchpanel display on which the user interface is displayed.

7. The system of claim 1, wherein the external monitor further comprises a video camera.

8. The system of claim 6, wherein the user interface is a graphical user interface that operates on the touchpanel display, the graphical user interface comprising a series of icons configured to control the computer and video interfaces.

9. The system of claim 1, wherein the video interface comprises an interface to a video conferencing service.

10. The system of claim 1, wherein the video interface comprises an interface to receive a television broadcast signal.

11. The system of claim 1, wherein the video interface comprises an interface to a service that provides on-demand movies.

12. A system for monitoring a patient's vital signs, the system for use with a an external video display component that includes a display device, a monitor interface, a computer interface for connecting to a computer network, a video interface for connecting to at least one other source for video content, and a processor programmed to respond to control information, said system comprising:
a sensor unit to be worn on the patient's body to monitor vital signs of the patient, said sensor unit including a first wireless transceiver and a processor programmed to determine blood pressure information from the monitored vital signs of the patient and transmit the blood pressure information via the first wireless transceiver; and
an external monitor including a monitor display device, a wireless transceiver for receiving the blood pressure information from the sensor unit, and a processor programmed to format the blood pressure information for display by the external video display component and further programmed to display on the monitor display device a user interface through which the patient generates the control information for controlling the external video display component to select whatever one or more of the monitor interface, the computer interface and the video interface to provide information to be displayed on the display device of the external video component.
